SEQUENCE LISTING (4 sequences)

SEQ ID NO: 1 - DNA, 20 bp, synthetic construct
  source 1..20: mol_type = other DNA
tataggctgggcttcccctt

SEQ ID NO: 2 - DNA, 20 bp, synthetic construct
  source 1..20: mol_type = other DNA
agctttctggtgtgactcgg

SEQ ID NO: 3 - DNA, 22 bp, synthetic construct
  source 1..22: mol_type = other DNA
gtggtctcctctgacttcaaca

SEQ ID NO: 4 - DNA, 22 bp, synthetic construct
  source 1..22: mol_type = other DNA
ctcttcctcttgtgctcttgct

(19) United States
(12) Patent Application Publication
(10) Pub. No.: US 2026/0151327 A1
(43) Pub. Date: Jun. 4, 2026

(54) COSMETIC COMPOSITION FOR SKIN PROTECTION CONTAINING SCUTELLARIA BAICALENSIS EXTRACT AS ACTIVE INGREDIENT

(71) Applicant: EVERBIO CO., LTD., Seoul (KR)

(72) Inventors: Boyong KIM, Gyeonggi-do (KR); Jae Hyeok JEONG, Gyeonggi-do (KR); Gyu Bin KONG, Gyeonggi-do (KR)

(21) Appl. No.: 19/125,571

(22) PCT Filed: Nov. 6, 2023
(86) PCT No.: PCT/KR2023/017685

§ 371 Date: Apr. 29, 2025

(30) Foreign Application Priority Data
Nov. 4, 2022 (KR) 10-2022-0146550
Mar. 21, 2023 (KR) 10-2023-0036967

(51) Int. Cl. A61K 8/9789; A61K 36/539; A61P 17/10; A61Q 17/00; A61Q 19/00

(52) U.S. Cl. CPC A61K 8/9789; A61K 36/539; A61P 17/10; A61Q 17/00; A61Q 19/008

(57) ABSTRACT

The present invention relates to a cosmetic composition for skin protection containing a Scutellaria baicalensis extract as an active ingredient. The Scutellaria baicalensis extract of the present invention protects the skin from harmful substances brought by air pollution and has the effects of anti-inflammation and sebum secretion inhibition, and thus may be usefully applied in a cosmetic product for skin protection or anti-pollution.

### TECHNICAL FIELD

This application claims priority to and the benefit of Korean Patent Application No. 10-2022-01465502022, filed in the Korean Intellectual Property Office on Nov. 4, 2022, and Korean Patent Application No. 10-2023-0036967, filed in the Korean Intellectual Property Office on Mar. 21, 2023, the disclosures of which are incorporated herein by reference.

The present disclosure relates to a cosmetic composition for skin protection containing a Scutellaria baicalensis extract as an active ingredient.

### BACKGROUND ART

The skin, an organ located on the outermost part of the body, serves as an essential barrier that protects the human body by retaining moisture in the body and preventing invasion factors from the outside. As environmental pollution has worsened due to the industrialization of modern society, the skin of modern people is exposed to harmful substances resulting from environmental pollution, especially, air pollution, over a long period of time. The long-term exposure of the skin to the harmful substances may result in disorders in the skin barrier, causing skin damage or skin diseases. The causes of air pollution that adversely affect the skin are factory smoke, automobile exhaust, particulate matter (PM), fine particulate matter, and the like. Particularly, particulate matter refers to a particulate material containing chemicals derived from industrial hazardous substances, which were designated as Group 1 carcinogens by the World Health Organization (WHO) in 2013, and indicates a particulate material with a diameter of 10 μm or less (PM10), while fine particulate matter refers to a particulate material having a size of 2.5 μm or less (PM2.5). Particulate matter and fine particulate matter contain chemical components, such as nitrates, sulfates, and polycyclic aromatic hydrocarbons (PAHs), and heavy metals, such as lead and cadmium.

Benzo[a]pyrene, one of the polycyclic aromatic hydrocarbons, has been designated as a Group 1 carcinogen (a substance clearly established to have a correlation with cancer development) by the International Agency for Research on Cancer (IARC) under the WHO. Benzo[a]pyrene is not only contained in automobile exhaust, diesel gas, wood combustion products, plastic combustion products, and cigarette smoke, but also generated even during food processing, and thus people are easily exposed to the compound even in daily life. Benzo[a]pyrene, upon the contact with the skin, causes various skin lesions by exhibiting toxicity, inducing an inflammation response, damaging DNA to result in cancer development, and the like. The lesions caused by benzo[a]pyrene weaken the skin's metabolic functions as well as impair sebum regulation functions, thereby causing excessive sebum secretion. Such excessive sebum secretion may accelerate skin aging by inducing the expansion of pores, which are discharge channels of sebum and waste, to weaken the ability to synthesize elastin and collagen, result in acne by filling the openings of pores due to the solidification of sebum in the pores, or cause seborrheic dermatitis, a challenging eczematous condition, resulting from increased sebum secretion.

Steroidal anti-inflammatory preparations are used for skin lesions or skin diseases caused by environmental pollution, but steroidal preparations have side effects, such as skin atrophy, stretch marks, skin ulcers, and infections, and thus the use thereof is limited. Hence, there is an urgent need to develop a preparation, that is, an anti-pollution preparation that can protect the skin from air pollutants while having fewer side effects and low toxicity to the human body.

Throughout the specification, many papers and patent documents are referenced and their citations are provided. The disclosures of cited papers and patent documents are entirely incorporated by reference herein, and the level of the technical field within which the present disclosure falls and details of the present disclosure are explained more clearly.

### DISCLOSURE OF INVENTION

### Technical Problem

The present inventors made intensive efforts to develop a cosmetic composition that can protect the skin from harmful substances due to air pollution while having fewer side effects and low toxicity to the human body. As a result, the present inventors established that a Scutellaria baicalensis extract protects the skin from harmful substances due to air pollution, alleviates skin inflammation caused by harmful substances, and suppresses sebum secretion, and thus completed the present disclosure.

Accordingly, an aspect of the present disclosure is to provide a cosmetic composition for skin protection.

Other aspects and advantages of the present disclosure will become more obvious when taken with the following detailed description, claims, and drawings of the present disclosure.

### Solution to Problem

In accordance with an aspect of the present disclosure, a cosmetic composition for skin protection is provided containing a Scutellaria baicalensis extract as an active ingredient.

The present inventors made intensive efforts to develop a cosmetic composition that can protect the skin from harmful substances due to air pollution while having fewer side effects and low toxicity to the human body. As a result, the present inventors established that a Scutellaria baicalensis extract protects the skin from harmful substances, alleviates skin inflammation caused by harmful substances, and suppresses sebum secretion.

Herein, Scutellaria baicalensis is a perennial herb belonging to the Lamiaceae family in the Lamiales order, widely distributed in mountain regions throughout Korea, and also called Jageum ( [symbol] ), Jogeum ( [symbol] ), and Pyeongeum ( [symbol] ). The young shoots are edible, and the roots are medically used due to their hemostatic properties and the like, and traditionally, has been used as an herbal medicine for infectious hepatitis, diuresis, anti-inflammatory fever, hemorrhagic inflammation, and the like. The Scutellaria baicalensis herein may be raw Scutellaria baicalensis, a crushed product of the herbal medicine, a dried product of the herbal medicine, or a crushed, dried product of the herbal medicine, and most specifically, may be a dried product obtained by hot-air drying Scutellaria baicalensis, but is not limited thereto. Scutellaria baicalensis used herein may be obtained without limitation, and may be used through cultivation or by purchase from the market.

The Scutellaria baicalensis extract used in the composition of the present disclosure may be an extract of a portion or the whole of the aerial or root part of the herbaceous plant of Scutellaria baicalensis. More specifically, the Scutellaria baicalensis extract used in the composition of the present disclosure is at least one extract obtained from the leaves, flowers, stems, fruits, roots, or a combination thereof of the herbaceous plant of Scutellaria baicalensis, and most specifically, is an extract of roots of Scutellaria baicalensis, but is not limited thereto.

In an embodiment of the present disclosure, the Scutellaria baicalensis extract used in the composition of the present disclosure is an extract obtained by extraction from the above-described dried product obtained by drying Scutellaria baicalensis. More specifically, the drying may be conducted by spray drying, hot-air drying, freeze drying, natural drying, or vacuum drying, but is not limited thereto.

In an embodiment of the present disclosure, the Scutellaria baicalensis extract used in the composition of the present disclosure is an extract obtained by extraction from the above-described dried product obtained by hot-air drying Scutellaria baicalensis. More specifically, the hot-air drying may be performed at 25° C. to 150° C., and more specifically 25° C. to 150° C., 25° C. to 120° C., 25° C. to 100° C., 25° C. to 85° C., 25° C. to 70° C., 45° C. to 150° C., 45° C. to 120° C., 45° C. to 100° C., 45° C. to 85° C., 45° C. to 70° C., 60° C. to 150° C., 60° C. to 120° C., 60° C. to 100° C., 60° C. to 85° C., 60° C. to 70° C., 70° C. to 150° C., 70° C. to 120° C., 70° C. to 100° C., or 70° C. to 85° C., but is not limited thereto.

The Scutellaria baicalensis extract used in the composition of the present disclosure may be obtained by purchase or direct extraction. In cases where the Scutellaria baicalensis extract used in the composition of the present disclosure is obtained by treating Scutellaria baicalensis with an extraction solvent, various extraction solvents, such as a polar solvent or a non-polar solvent, may be used. Suitable examples of the polar solvent include (i) water, (ii) an alcohol (preferably, methanol, ethanol, propanol, butanol, normal-propanol, iso-propanol, normal-butanol, 1-pentanol, 2-butoxyethanol, or ethylene glycol), (iii) acetic acid, (iv) dimethyl-formamide (DMFO), and (v) dimethyl sulfoxide (DMSO). Suitable examples of the non-polar solvent include acetone, acetonitrile, ethyl acetate, methyl acetate, fluoroalkanes, pentane, hexane, 2,2,4-trimethylpentane, decane, cyclohexane, cyclopentane, diisobutylene, 1-pentene, 1-chlorobutane, 1-chloropentane, o-xylene, diisopropyl ether, 2-chloropropane, toluene, 1-chloropropane, chlorobenzene, benzene, diethyl ether, diethyl sulfide, chloroform, dichloromethane, 1,2-dichloroethane, aniline, diethyl amine, ether, carbon tetrachloride, and tetrahydrofuran (THF).

In an embodiment of the present disclosure, the Scutellaria baicalensis extract may be obtained by extraction with any one selected from the group consisting of water, an organic solvent, and a mixture solvent thereof.

In an embodiment of the present disclosure, the organic solvent may be at least one selected from the group consisting of a C1 to C4 lower alcohol, petroleum ether, hexane, benzene, chloroform, methylene chloride, an ether, ethyl acetate, and acetone, but is not limited thereto.

The extraction temperature for the extract of the present disclosure may be, but is not limited to 0° C. to 140° C., and specifically, may be 0° C. to 140° C., 0° C. to 120° C., 0° C. to 100° C., 0° C. to 85° C., 0° C. to 75° C., 0° C. to 70° C., 20° C. to 140° C., 20° C. to 120° C., 20° C. to 100° C., 20° C. to 85° C., 20° C. to 75° C., 20° C. to 70° C., 40° C. to 140° C., 40° C. to 120° C., 40° C. to 100° C., 40° C. to 85° C., 40° C. to 75° C., 40° C. to 70° C., 55° C. to 140° C., 55° C. to 120° C., 55° C. to 100° C., 55° C. to 85° C., 55° C. to 75° C., 55° C. to 70° C., 65° C. to 140° C., 65° C. to 120° C., 65° C. to 100° C., 65° C. to 85° C., 65° C. to 75° C., 65° C. to 70° C., 70° C. to 140° C., 70° C. to 120° C., 70° C. to 100° C., 70° C. to 85° C., or 70° C. to 75° C., but is not limited thereto.

The extraction time for the extract of the present disclosure is not particularly limited, and for example, the extraction time may be 30 minutes to 10 days, 30 minutes to 5 days, 30 minutes to 48 hours, 30 minutes to 36 hours, 30 minutes to 27 hours, 30 minutes to 24 hours, 3 hours to 10 days, 3 hours to 5 days, 3 hours to 48 hours, 3 hours to 36 hours, 3 hours to 27 hours, 3 hours to 24 hours, 9 hours to 10 days, 9 hours to 5 days, 9 hours to 48 hours, 9 hours to 36 hours, 9 hours to 27 hours, 9 hours to 24 hours, 15 hours to 10 days, 15 hours to 5 days, 15 hours to 48 hours, 15 hours to 36 hours, 15 hours to 27 hours, 15 hours to 24 hours, 21 hours to 10 days, 21 hours to 5 days, 21 hours to 48 hours, 21 hours to 36 hours, 21 hours to 27 hours, or 21 hours to 24 hours, but is not limited thereto.

In an embodiment of the present disclosure, the Scutellaria baicalensis extract may be contained in a content of 0.01 wt % to 30 wt % relative to the total weight of the composition. More specifically, the Scutellaria baicalensis extract may be, relative to the total weight of the composition, 0.01 wt % to 30 wt %, 0.01 wt % to 20 wt %, 0.01 wt % to 15 wt %, 0.01 wt % to 12.5 wt %, 0.01 wt % to 10 wt %, 0.1 wt % to 30 wt %, 0.1 wt % to 20 wt %, 0.1 wt % to 15 wt %, 0.1 wt % to 12.5 wt %, 0.1 wt % to 10 wt %, 1 wt % to 30 wt %, 1 wt % to 20 wt %, 1 wt % to 15 wt %, 1 wt % to 12.5 wt %, 1 wt % to 10 wt %, 5 wt % to 30 wt %, 5 wt % to 20 wt %, 5 wt % to 15 wt %, 5 wt % to 12.5 wt %, 5 wt % to 10 wt %, 7.5 wt % to 30 wt %, 7.5 wt % to 20 wt %, 7.5 wt % to 15 wt %, 7.5 wt % to 12.5 wt %, or 7.5 wt % to 10 wt %, but is not limited thereto.

In the present disclosure, the Scutellaria baicalensis extract may be used in the form of a crude extract obtained by extraction with a solvent, and may be used through high-purity purification.

As used herein, the term “extract” has a meaning that is commonly used as a crude extract in the art as described above, and, broadly, encompasses a fraction obtained by additionally fractionating the extract. That is, examples of the Scutellaria baicalensis extract include ones obtained by using the above-described extraction solvents as well as ones obtained by additionally applying a purification procedure thereto. For example, fractions obtained by passing the extract through an ultrafiltration membrane with a predetermined molecular weight cut-off value or fractions obtained by additionally performing various purification methods, such as various types of chromatography (designed for separation according to size, charge, hydrophobicity, or hydrophilicity) are also included in the Scutellaria baicalensis extract of the present disclosure.

The Scutellaria baicalensis extract used in the present disclosure may be prepared in a powder form by additional procedures, such as distillation under reduced pressure and freeze-drying or spray drying.

In an embodiment of the present disclosure, the composition of the present disclosure may contain, in addition to the above-described Scutellaria baicalensis extract, ingredients that are commonly used in a cosmetic composition, and for example, may contain conventional additives, such as an antioxidant, a stabilizer, a solubilizer, a vitamin, a pigment, and a flavoring agent, and a carrier.

Examples of the carrier may include purified water, a monohydric alcohol (ethanol or propyl alcohol), a polyhydric alcohol (glycerol, 1,3-butylene glycol, or propylene glycol), a higher fatty acid (palmitic acid or linolenic acid), an oil (wheat germ oil, camellia oil, jojoba oil, olive oil, squalene, sunflower oil, macadamia peanut oil, avocado oil, soybean water-added lecithin, or fatty acid glyceride), and the like, but are not limited thereto. As needed, a surfactant, a sterilizer, an antioxidant, a UV absorber, an anti-inflammatory agent, and a refreshing agent may also be added.

Examples of the surfactant may include polyoxyethylene, hydrogenated castor oil, polyoxyethylene, oleyl ether, polyoxyethylene monooleate, polyoxyethylene, glyceryl monostearate, sorbitan monostearate, polyoxyethylene monooleate, sorbitan, sucrose fatty acid ester, hexaglycerin monolaurate, polyoxyethylene reduced lanolin, POE, glyceryl pyroglutamic acid, isostearic acid, diester, N-acetyl glutamine, isostearyl ester, and the like.

Examples of the sterilizer may include hinokitiol, triclosan, chlorhexidine gluconate, phenoxyethanol, resorcin, isopropylmethylphenol, azulene, salicylic acid, zinc pyrithione, and the like.

Any of butyl hydroxy anisole, gallic acid, propyl gallate, and erythorbic acid may be used as the antioxidant.

Examples of the ultraviolet absorbent may include benzophenones such as dihydroxybenzophenone, melanin, ethyl para-aminobenzoate, 2-ethylhexyl para-dimethyl aminobenzoate, cinoxate, 2-ethylhexyl p-methoxycinnamate, 2-(2-hydroxy-5-methylphenyl)benzotriazole, urocanic acid, metal oxide particles, and the like.

Examples of the anti-inflammatory agent may include dipotassium glycyrrhizinate, allantoin, and the like, and examples of the refreshing agent may include capsicum tincture, 1-menthol, and the like.

In another embodiment of the present disclosure, the cosmetic composition of the present disclosure may be formulated in a dosage form selected from the group consisting of a solution, a topical ointment, a gel, a cream, a foam, a nourishing toner, a softening toner, a pack, a softening lotion, an emulsion, a makeup base, an essence, a skin ampoule, a hair ampoule, a scalp treatment, a hair tonic, a hair conditioner, a hair treatment, a hair lotion, a hair shampoo, a hair rinse, a conditioner shampoo, a hair nourishing toner, a hair gel, a hair wax, a hair spray, a hair dye, a soap, a liquid cleanser, a bath additive, a sunscreen cream, a sun oil, a suspension, an emulsion, a paste, a skin lotion, a powder, a skin cleansing, a powder foundation, an emulsion foundation, a wax foundation, a patch, and a spray, but is not limited thereto.

In an embodiment of the present disclosure, the above-described cosmetic composition for skin protection of the present disclosure has an anti-pollution effect on harmful substances due to air pollution, but is not limited thereto.

Herein, the harmful substances due to air pollution mean materials that harm human health or property or negatively impact natural environments including animal and plant growth environments, such as smoke, dust, gases, and odors. Examples of the harmful substances due to air pollution include factory smoke, automobile exhaust, particulate matter, fine particulate matter, and the like. The particulate matter (PM) refers to a particulate material containing chemicals derived from industrial hazardous substances, which were designated as Group 1 carcinogens by the World Health Organization (WHO) in 2013, indicating a particulate material with a diameter of 10 μm or less (PM10), while the fine particulate matter refers to a particulate material having a size of 2.5 μm or less (PM2.5). The particulate matter and the fine particulate matter contain chemical components, such as nitrates, sulfates, and polycyclic aromatic hydrocarbons (PAHs), and heavy metals, such as lead and cadmium.

As used herein, the term “anti-pollution effect” refers to an effect of soothing and protecting the skin sensitized by the stress, such as harmful substances, due to an environmental pollution, especially, air pollution, and the stimulation of environmental factors in daily life.

As validated in a specific embodiment of the present disclosure, the cosmetic composition of the present disclosure increases the cellular viability in adipose-derived stem cells treated with benzo[a]pyrene, leading to an anti-pollution effect of protecting the skin from harmful substances due to air pollution. Therefore, the composition for skin protection of the present disclosure can be utilized as a composition for anti-pollution.

In an embodiment of the present disclosure, the above-described cosmetic composition for skin protection of the present disclosure has an anti-inflammatory effect. As validated in a specific embodiment of the present disclosure, the cosmetic composition of the present disclosure significantly alleviated the skin inflammation when applied to the skin.

In an embodiment of the present disclosure, the above-described cosmetic composition for skin protection of the present disclosure has a sebum secretion suppressing effect. In a specific embodiment of the present disclosure, the cosmetic composition of the present disclosure, when applied to the skin, reduces the skin sebum content to exhibit a sebum secretion suppressing effect, leading to less skin oiliness.

In an embodiment of the present disclosure, the above-described cosmetic composition for skin protection of the present disclosure suppresses sebum secretion by promoting lipolysis. As validated in a specific embodiment of the present disclosure, the cosmetic composition of the present disclosure has effects of suppressing sebum secretion by promoting the synthesis of AMP-activated protein kinase (AMPK) and hormone sensitive lipase (HSL), the most common lipases, in adipose-derived stem cells, and promoting lipolysis by the enzymes. Particularly, the cosmetic composition for skin protection of the present disclosure, even when used to treat together with benzo[a]pyrene together, efficiently promotes lipolysis, thereby exhibiting an effect of suppressing excessive sebum secretion caused by harmful substances due to air pollution.

In an embodiment of the present disclosure, the above-described cosmetic composition for skin protection of the present disclosure inhibits lipid synthesis to suppress sebum secretion. As validated in a specific embodiment of the present disclosure, the cosmetic composition of the present disclosure has an effect of inhibiting lipid synthesis by inhibiting the expression of CCAAT enhancer binding protein alpha (CEBP-α), one of the lipid synthesis promoting genes, in adipose-derived stem cells.

In an embodiment of the present disclosure, the above-described cosmetic composition for skin protection of the present disclosure can alleviate a disease caused by excessive sebum secretion. The above-described disease may be acne or seborrheic dermatitis, but is not limited thereto.

Herein, acne is a chronic inflammatory disease developed in the sebaceous glands attached to hair follicles, and acne frequently occurs on oily areas of the skin, such as the face, neck, back, and chest, and usually develops during adolescence but may also occur in adults. Acne is known to occur by a composite action of genetic factors, environmental factors, follicular responsiveness, and the like, and the main causes include an increase in sebum secretion, an excess of skin keratinization, and an induction of inflammation by the proliferation of Propionibacterium acne.

As used herein, seborrheic dermatitis, which is a chronic eczematous disease caused by an increase in sebum secretion, frequently occurs in areas with high sebum secretion, such as the scalp, forehead, and armpits, and has main symptoms, such as erythema (red spots) and fine scaling (dandruff).

The cosmetic composition for skin protection of the present disclosure suppresses sebum secretion, which is a cause of acne and seborrheic dermatitis, and thus shows excellent effects in the prevention and alleviation of acne or seborrheic dermatitis.

In an embodiment of the present disclosure, the above-described cosmetic composition for skin protection of the present disclosure may contain the Scutellaria baicalensis extract of the present disclosure at 1% (v/v) to 30% (v/v). More specifically, the above-described cosmetic composition for skin protection of the present disclosure may contain the Scutellaria baicalensis extract of the present disclosure at 1% (v/v) to 30% (v/v), 1% (v/v) to 25% (v/v), 1% (v/v) to 20% (v/v), 1% (v/v) to 17.5% (v/v), 1% (v/v) to 15% (v/v), 1% (v/v) to 12.5% (v/v), 1% (v/v) to 10% (v/v), 5% (v/v) to 30% (v/v), 5% (v/v) to 25% (v/v), 5% (v/v) to 20% (v/v), 5% (v/v) to 17.5% (v/v), 5% (v/v) to 15% (v/v), 5% (v/v) to 12.5% (v/v), 5% (v/v) to 10% (v/v), 7.5% (v/v) to 30% (v/v), 7.5% (v/v) to 25% (v/v), 7.5% (v/v) to 20% (v/v), 7.5% (v/v) to 17.5% (v/v), 7.5% (v/v) to 15% (v/v), 7.5% (v/v) to 12.5% (v/v), or 7.5% (v/v) to 10% (v/v), but is not limited thereto.

In accordance with still another aspect of the present disclosure, a skin protection method is provided including applying an effective amount of the above-described cosmetic composition for skin protection of the present disclosure to the skin.

As used herein, the term “effective amount” refers to an amount that is sufficient to achieve skin protective efficacy, anti-inflammation efficacy, and sebum secretion suppressing efficacy of the above-described composition of the present disclosure.

The skin protection method of the present disclosure is implemented by using the above-described cosmetic composition for skin protection according to an aspect of the present disclosure, and therefore, the overlapping contents of both disclosures are applied in the same manner and the description of the present specification will be omitted to avoid the complexity of the specification.

### Advantageous Effects of Invention

The features and advantages of the present disclosure are summarized as follows.

(a) The present disclosure provides a cosmetic composition for skin protection.

(b) The cosmetic composition for skin protection of the present disclosure protects the skin from harmful substances due to air pollution and has an anti-inflammation effect and a sebum secretion suppressing effect, and thus can be advantageously used as a cosmetic product for skin protection or anti-pollution.

### BRIEF DESCRIPTION OF DRAWINGS

FIG. 1 shows cellular viability of adipose-derived stem cells (ASCs) exposed to benzo[a]pyrene and a Scutellaria baicalensis extract (Con representing control and ns representing a non-significant difference, *P<0.05, **P<0.01, ***P<0.001)

FIG. 2 shows Annexin-V-PI staining and flow cytometry analysis results of the protective effect of a Scutellaria baicalensis extract against the toxicity of benzo[a]pyrene (*P<0.05, **P<0.01, ***P<0.001)

FIG. 3A shows staining and flow cytometry analysis results of the lipolysis promoting effect of a Scutellaria baicalensis extract.

FIG. 3B shows analysis results of the lipolysis promoting effect through synthesis amounts of AMP-Kinase (AMPK) and hormone sensitive lipase (HSL) (*P<0.05, ***P<0.001).

FIG. 4 shows immunochemical analysis results of the lipolysis promoting effect of a Scutellaria baicalensis extract (red: AMPK-stained cells, green: HSL-stained cells).

FIG. 5 shows analysis results of the effects of benzo[a]pyrene and a Scutellaria baicalensis extract on the mRNA level of CEBP-α, one of the lipid synthesis promoting genes (*P<0.05, ***P<0.001).

FIG. 6A shows comparison results of the subjects' perceived degree of skin inflammation before and after the use of a 10% (v/v) Scutellaria baicalensis extract essence.

FIG. 6B shows heatmap comparison results of 20 subjects' perceived degree of skin inflammation before and after the use of a 10% (v/v) Scutellaria baicalensis extract essence, compared with control.

FIG. 6C shows comparison results of the subjects' perceived degree of sebum secretion before and after the use of a 10% (v/v) Scutellaria baicalensis extract essence.

FIG. 6D shows heatmap comparison results of 20 subjects' perceived degree of sebum secretion before and after the use of a 10% (v/v) Scutellaria baicalensis extract essence, compared with control.

FIG. 6E shows comparison results of the subjects' perceived degree of skin condition before and after the use of a 10% (v/v) Scutellaria baicalensis extract essence.

FIG. 6F shows heatmap comparison results of 20 subjects' perceived degree of skin condition before and after the use of a 10% (v/v) Scutellaria baicalensis extract essence, compared with control.

FIG. 7 shows visual analysis results of the subjects' skin change before and after the use of a 10% (v/v) Scutellaria baicalensis extract essence, as obtained through photography.

FIGS. 8A, 8B, 8C, 8D, and 8E show analysis results of the subjects' skin changes before and after the use of a 10% (v/v) Scutellaria baicalensis extract essence, as obtained using a magnifier lens.

FIG. 9 shows measurement results of the subjects' skin sebum contents before and after the use of a 10% (v/v) Scutellaria baicalensis extract essence.

### MODE FOR CARRYING OUT THE INVENTION

Hereinafter, the present disclosure will be described in more detail with reference to exemplary embodiments. These exemplary embodiments are provided only for the purpose of specifically illustrating the present disclosure, and therefore, according to the purpose of the present disclosure, it would be apparent to a person skilled in the art that these exemplary embodiments are not construed to limit the scope of the present disclosure.

### Examples

(Throughout the present specification, the “%” used to express the concentration of a specific material, unless otherwise particularly stated, refers to (wt/wt) % for solid/solid, (wt/vol) % for solid/liquid, and (vol/vol) % for liquid/liquid.)

Preparation Example 1: Preparation of Scutellaria baicalensis Extract

Scutellaria baicalensis cultivated in Uiseong, Gyeongsangbuk-do, was purchased and used as an experimental material.

After the roots of Scutellaria baicalensis were hot-air dried at 70° C. for 24 hours and then crushed, the crushed product was subjected to extraction with distilled water at 70° C. and subsequently concentrated under reduced pressure by using a digital rotary evaporator (DAIHAN). The concentrated Scutellaria baicalensis extract was sterilized using a 0.22 μm microporous membrane (Merck) before use.

Example 1: Evaluation of Cytotoxicity of Scutellaria baicalensis Extract

### Example 1-1: Culture of Adipose Stem Cells

PCS-500-011™ cells (Thermo-Fisher Scientific, USA) were purchased as adipose-derived stem cells (ASCs) used in the experiment, from American Type Culture Collection (ATCC), and subcultured for 3 passages in the MesenPRO RS™ medium and MesenPRO RS™ growth supplement medium (Thermo-Fisher scientific, USA) until the live cells reached 80% to 90% confluency.

Specifically, the cell stock vial was thawed in a 37° C. water bath and suspended in a medium with an equal volume of the cell stock vial, and then the suspension was transferred to a tube and centrifuged (2,000 rpm, 2 min, 4° C.), followed by removal of the supernatant. The phosphate buffered saline (PBS) was added to the cell pellets remaining after the removal of the supernatant, and then the above procedures were repeated three times. Thereafter, the resultant product was placed in a medium where the cell pellets were to be grown, followed by gentle pipetting up and down, thereby preparing a cell suspension. The cell suspension was dispensed into a flask or dish containing an appropriate amount of medium. The cells were subcultured when the cells reached 70% to 80% confluency on the bottom.

PBS, media, and trypsin-EDTA were warmed at 37° C. for 30 minutes before use. The supernatant medium was removed by suction, and PBS was dispensed. After 2 mL to 3 mL of trypsin-EDTA was added, followed by incubation for 2 to 3 minutes in an incubator. The suspended cells were transferred to a tube and centrifuged at 2,000 rpm for 2 minutes at 4° C., and then the supernatant was removed. PBS was added to the cell pellets remaining after the removal of the supernatant, and then the same procedure was repeated five times. The cell pellets remaining after the removal of the supernatant were placed in a medium where the cell pellets were to be cultured, followed by gentle pipetting up and down. The cell suspension was dispensed into a flask or dish containing an appropriate amount of medium, followed by subculture for several passages and washing, thereby preparing cell pellets. The washed cell pellets were suspended in a medium containing FBS(4):DMSO(1):10% FBS and 1% penicillin-streptomycin, and the resultant suspension was aliquoted into 1 ml vials to prepare cell stocks, and then as needed, the vials were thawed for use.

Example 1-2: Evaluation of Cytotoxicity of Scutellaria baicalensis Extract

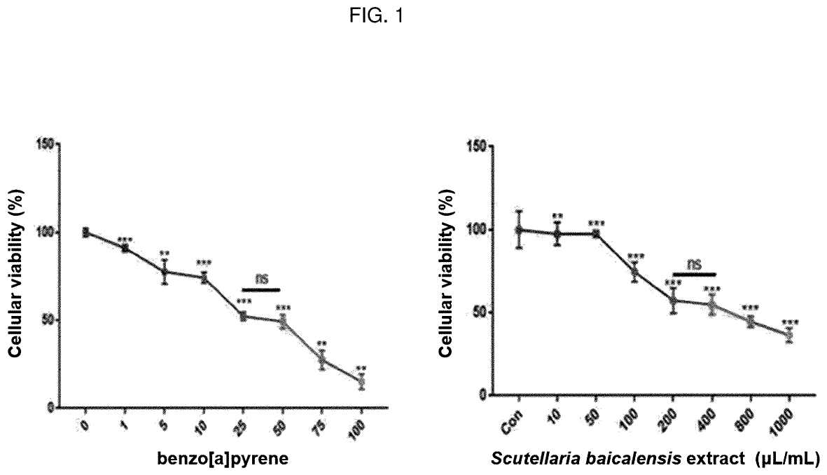

To establish the exposure concentrations of benzo[a]pyrene (BP) and the Scutellaria baicalensis extract on the adipose-derived stem cells (ASCs), the activity at which 50% of the adipose-derived stem cells survive. Benzo[a]pyrene was prepared with concentrations of 0 μM/mL, 1 μM/mL, 5 μM/mL, 10 μM/mL, 25 μM/mL, 50 μM/mL, 75 μM/mL, and 100 μM/mL, and the Scutellaria baicalensis extract (SBE) was prepared with concentrations of 0 μL/mL, 10 μL/mL, 50 μL/mL, 100 μL/mL, 200 μL/mL, 400 L/mL, 800 μL/mL, and 1000 μL/mL. The cultured adipose-derived stem cells were exposed to each concentration for 3 days, and then the cellular viability at the 50% cytotoxicity concentration (CC₅₀) was measured, and the results are plotted in FIG. 1.

As a result, as shown in FIG. 1, benzo[a]pyrene showed CC₅₀ at 25 μM/mL and 50 μM/mL, and the Scutellaria baicalensis extract showed CC₅₀ at 200 μL/mL and 400 μL/mL. As a result of identifying the effective concentration (EC₅₀), benzo[a]pyrene was determined to use a concentration of 50 μM/mL for subsequent experiments, and the Scutellaria baicalensis extract was determined to use two concentrations of 200 μL/mL and 400 μL/mL.

Example 2: Analysis of Adipose-Derived Stem Cell (ASC) Protective Effect of Scutellaria baicalensis Extract

The adipose-derived stem cell (ASC) protective effect of the Scutellaria baicalensis extract against the cell toxicity of benzo[a]pyrene was analyzed as below.

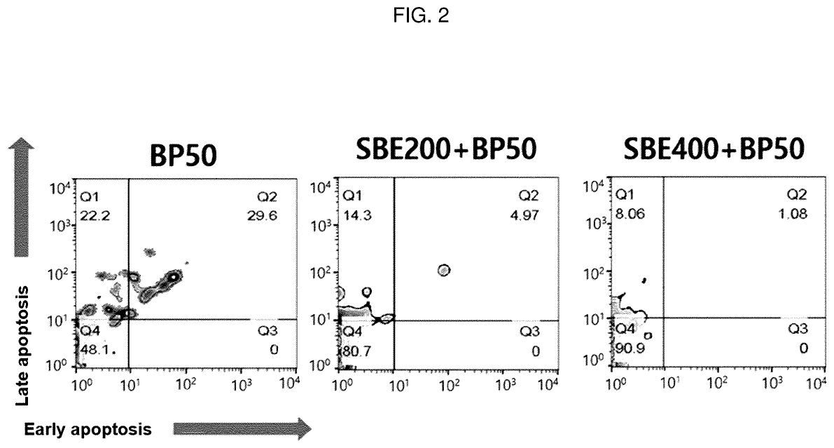

The adipose-derived stem cells cultured by the method in Example 1-1 were divided into i) a group treated with 50 μM/mL benzo[a]pyrene (BP50), ii) a group treated with 50 μM/mL benzo[a]pyrene and 200 μL/mL Scutellaria baicalensis extract (SBE200+BP50), and iii) a group treated with 50 μM/mL benzo[a]pyrene and 400 μL/mL Scutellaria baicalensis extract (SBE400+BP50). To observe cell apoptosis, the cells were stained using Annexin-V-PI dye kit (Invitrogen, USA). Since Annexin-V binds to phosphatidylserine (PS), which was exposed on the outer cell membrane during early apoptosis, early apoptosis can be identified using Annexin-V. Meanwhile, propidium iodide (PI), which binds to the nucleus, stains the nucleus through holes formed in the cell membrane in a state of late apoptosis or necrosis, thereby allowing the identification of late apoptotic or dead cells. Upon the treatment with recombinant Annexin-V conjugated to green fluorescent fluorescein isothiocyanate (FITC) and red fluorescent PI, apoptotic cells showed green fluorescence, dead cells showed red and green fluorescence, and live cells showed almost no fluorescence. For result analysis, the cellular viability was measured using a flow cytometer (BD FACSCalibur) and FlowJo 10.6.1 (BD Bioscience), and the results are shown in FIG. 2.

As a result, as shown in FIG. 2, the group treated with 50 μM/mL benzo[a]pyrene (BP50) showed a cellular viability of 48.1%, early apoptotic cell proportion of 29.6%, and a late apoptotic cell proportion of 51.8%. The group treated with 50 μM/mL benzo[a]pyrene and 200 μL/mL Scutellaria baicalensis extract (SBE200+BP50) and the group treated with 50 μM/mL benzo[a]pyrene and 400 μL/mL Scutellaria baicalensis extract (SBE400+BP50) reached cell viabilities of 80.7% and 90.9%, respectively, indicating 1.68 times and 1.89 times the cell viabilities of the treatment with benzo[a]pyrene alone. These results confirmed that the Scutellaria baicalensis extract exhibited an effect of protecting adipose-derived stem cells from the cytotoxicity of benzo[a]pyrene.

Example 3: Analysis of Lipolysis Promoting Effect of Scutellaria baicalensis Extract

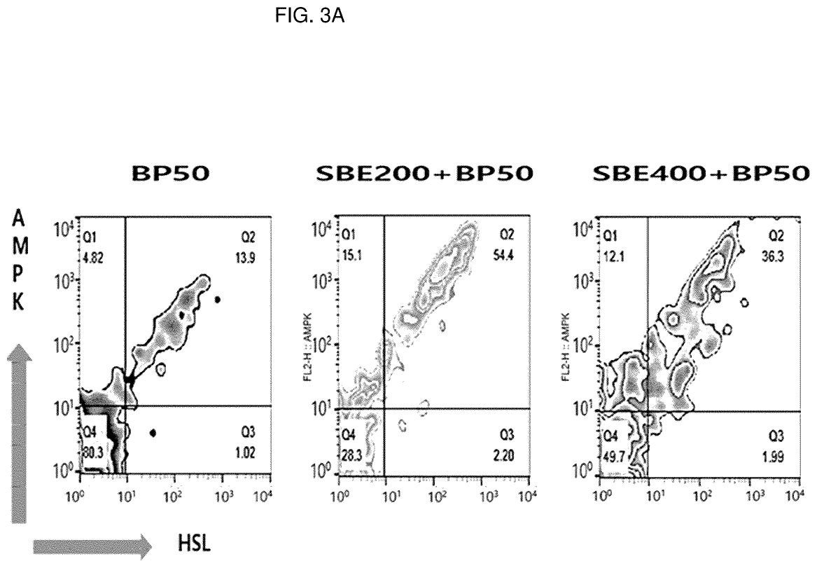

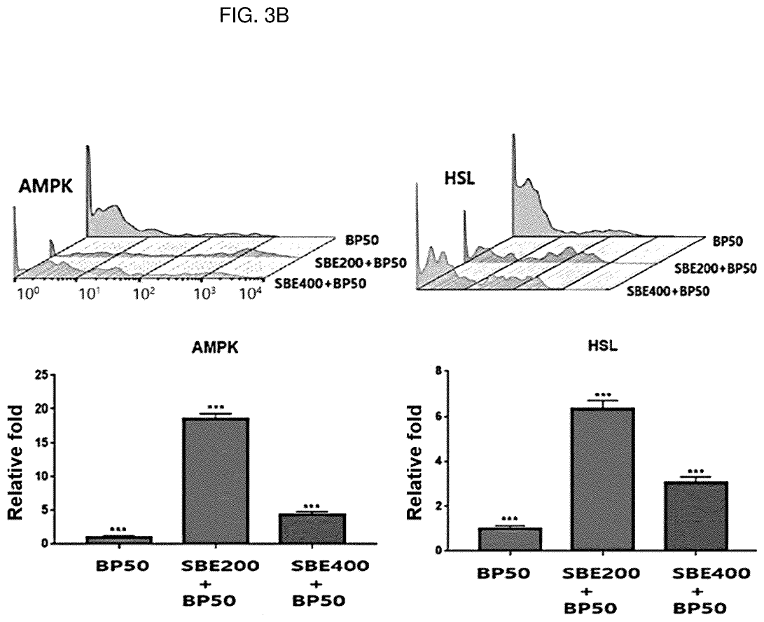

AMP-activated protein kinase (AMPK) and hormone sensitive lipase (HSL), most common lipolytic enzymes, were applied to adipose-derived stem cells (ASCs), and the lipids obtained by degradation through AMPK were stained with PE-anti-AMPK (R&D system, USA), and the lipids obtained by degradation through HSL were stained with FITC-anti-HSL (R&D system, USA) for 24 hours. The lipids were washed with PBS, and then analyzed using a flow cytometer (FACSCalibur) and FlowJo v10.6.1 (BD Bioscience, USA), and the results are shown in FIG. 3A.

As a result, as shown in FIG. 3A, upon exposure of the adipose-derived stem cells to 50 M/mL benzo[a]pyrene (BP50), the proportion of cells undergoing lipolysis by AMPK was 18.72% and the proportion of cells undergoing lipolysis by HSL was 14.92%. Upon the treatment with 50 μM/mL benzo[a]pyrene and 200 μL/mL Scutellaria baicalensis extract (SBE200+BP50), the proportion of cells undergoing lipolysis by AMPK was 69.5% and the proportion of cells undergoing lipolysis by HSL was 56.6%, which were 3.71-fold and 3.79-fold higher, respectively, compared with the treatment with 50 μM/mL benzo[a]pyrene (BP50) alone. Upon the treatment with 50 μM/mL benzo[a]pyrene and 400 L/mL Scutellaria baicalensis extract (SBE400+BP50), the proportion of cells undergoing lipolysis by AMPK was 48.4% and the proportion of cells undergoing lipolysis by HSL was 38.3%, which were higher, respectively, compared with the treatment with 50 μM/mL benzo[a]pyrene (BP50) alone.

The synthesis levels of AMPK and HSL were analyzed and plotted in FIG. 3B. As shown in FIG. 3B, the synthesis levels of AMPK and HSL were 18.21-fold and 6.23-fold higher in the treatment with 50 μM/mL benzo[a]pyrene and 200 μL/mL Scutellaria baicalensis extract (SBE200+BP50), compared with the treatment with 50 μM/mL benzo[a]pyrene alone, respectively, and 18.21-fold and 6.23-fold higher in the treatment with 50 μM/mL benzo[a]pyrene and 400 μL/mL Scutellaria baicalensis extract (SBE400+BP50), compared with the treatment with 50 M/mL benzo[a]pyrene (BP50) alone, respectively.

These results confirmed that the Scutellaria baicalensis extract exhibited a lipolysis promoting effect in adipose-derived stem cells.

Example 4: Immunochemical Analysis of Lipolysis Promoting Effect of Scutellaria baicalensis Extract

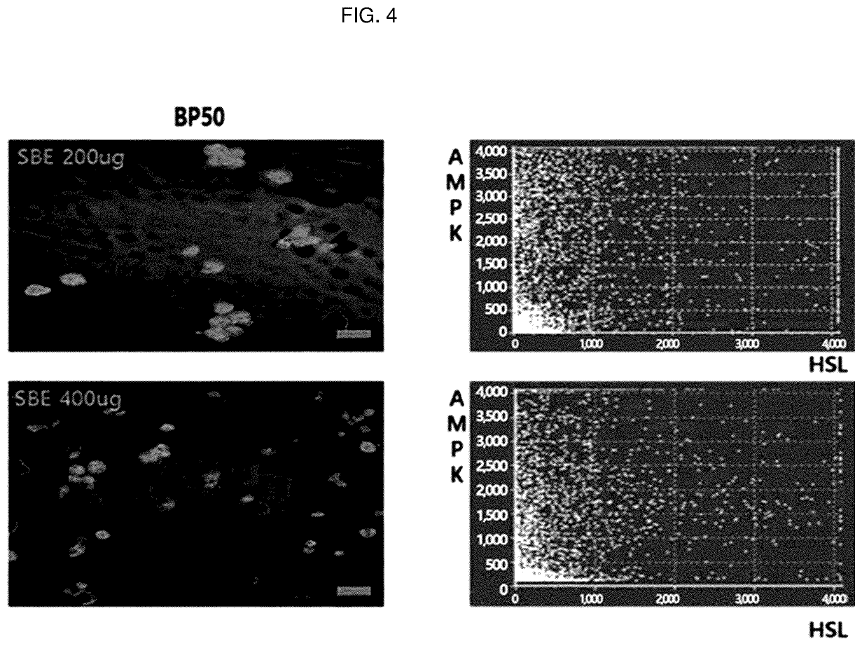

Adipose-derived stem cells (ASCs) were stained with the above-described antibodies PE-anti-AMPK and FITC-anti-HSL used in Example 4, and the fluorescence intensity and cell count were measured using a confocal microscope (FV-10, Olympus, Japan). The measurement results are shown in FIG. 4. The AMPK-treated cells appeared in red by PE-anti-AMPK, and the HSL-treated cells appeared in green by FITC-anti-HSL.

As shown in FIG. 4, the distribution of the stained cells was 1.5-fold higher in the treatment with 50 M/mL benzo[a]pyrene and 200 μL/mL Scutellaria baicalensis extract (SBE200+BP50) compared with the treatment with 50 μM/mL benzo[a]pyrene and 400 μL/mL Scutellaria baicalensis extract (SBE400+BP50), indicating that the treatment with 50 M/mL benzo[a]pyrene and 200 μL/mL Scutellaria baicalensis extract (SBE200+BP50) exhibited a higher lipolysis promoting effect.

Example 5: Analysis of Lipolysis Inhibitory Effect of Scutellaria baicalensis Extract

The influence of benzo[a]pyrene and the Scutellaria baicalensis extract on the mRNA expression level of CCAAT enhancer binding protein alpha (CEBP-α), one of the lipolysis promoting genes, was investigated by the method as below.

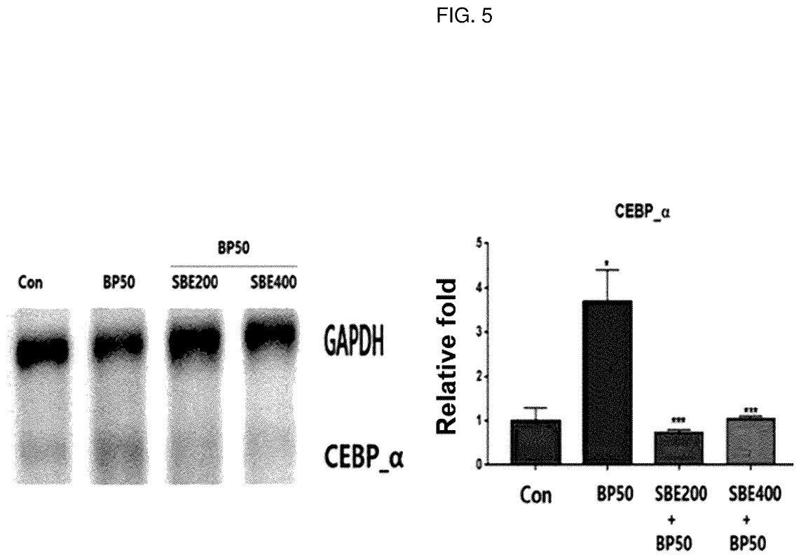

The adipose-derived stem cells were divided into i) a control group with no treatment (Con), ii) a group treated with 50 μM/mL benzo[a]pyrene (BP50), iii) a group treated with 50 μM/mL benzo[a]pyrene and 200 μL/mL Scutellaria baicalensis extract (SBE200+BP50), and iv) a group treated with 50 μM/mL benzo[a]pyrene and 400 μL/mL Scutellaria baicalensis extract (SBE400+BP50), and the total RNA of each cell group was extracted using Ribose reagent (GeneAll, Seoul, Korea). RNA was used to synthesize cDNA under the conditions of 1 minute at 95° C. and 35 seconds at 72° C. by using Maxime RT PreMix (iNtRON, Seungnam, Korea). The synthesized cDNA was amplified using the primers shown in Table 1 under the conditions of 15 seconds at 95° C., 30 seconds at 59° C., and 1 minute at 72° C. The amount of cDNA amplification was analyzed using iBright (FL1000, Thermo Fisher Scientific, Waltham, MA, USA) and iBright Analysis software 3.1.3 (Thermo Fisher Scientific, Waltham, MA, MA, USA), and the results are shown in FIG. 5.

TABLE 1

| Primer name | Primer sequence | SEQ ID NO |
|---|---|---|
| CEBP-α Forward | TATAGGCTGGGCTTCCCCTT | 1 |
| CEBP-α Reverse | AGCTTTCTGGTGTGACTCGG | 2 |
| GADPH Forward | GTGGTCTCCTCTGACTTCAACA | 3 |
| GADPH Reverse | CTCTTCCTCTTGTGCTCTTGCT | 4 |

As shown in FIG. 5, the mRNA expression level of CEBP-α was 4.73-fold higher in the group treated with 50 μM/mL benzo[a]pyrene (BP50) compared with the control group (Con). The mRNA expression level of CEBP-α was 0.71-fold lower in the group treated with 50 μM/mL benzo[a]pyrene and 200 μL/mL Scutellaria baicalensis extract (SBE200+BP50) compared with the control group, showing 6.66-fold lower compared with the group treated with 50 M/mL benzo[a]pyrene (BP50). The mRNA expression level of CEBP-α was 0.91-fold lower in the group treated with 50 μM/mL benzo[a]pyrene and 400 μL/mL Scutellaria baicalensis extract (SBE400+BP50) compared with the control group, showing 5.19-fold lower compared with the group treated with 50 μM/mL benzo[a]pyrene (BP50).

These results confirmed that the Scutellaria baicalensis extract exhibited a lipolysis inhibitory effect in adipose-derived stem cells.

Example 6: Test of Skin Application of Scutellaria baicalensis Extract

The skin application test was carried out to investigate the degrees of inflammation alleviation, sebum secretion suppression, and skin condition improvement of the Scutellaria baicalensis extract. A 10% Scutellaria baicalensis extract essence (hereinafter, “Scutellaria baicalensis extract essence”) was prepared by adding the Scutellaria baicalensis extract, glycerin, and PBS at the amounts shown in Table 2. The test was carried out from Nov. 3, 2021 to Nov. 18, 2021.

TABLE 2

| Ingredient | Content |
|---|---|
| Scutellaria baicalensis extract | 10 mL |
| Glycerin | 10 mL |
| PBS | Up to 100 mL |

After 20 men and women in their 20s with problematic skin including oily skin, acne, and the like were selected as subjects, the subjects underwent the application of the above-described Scutellaria baicalensis extract essence twice a day, in the morning and evening.

### Example 6-1. Sensory Evaluation

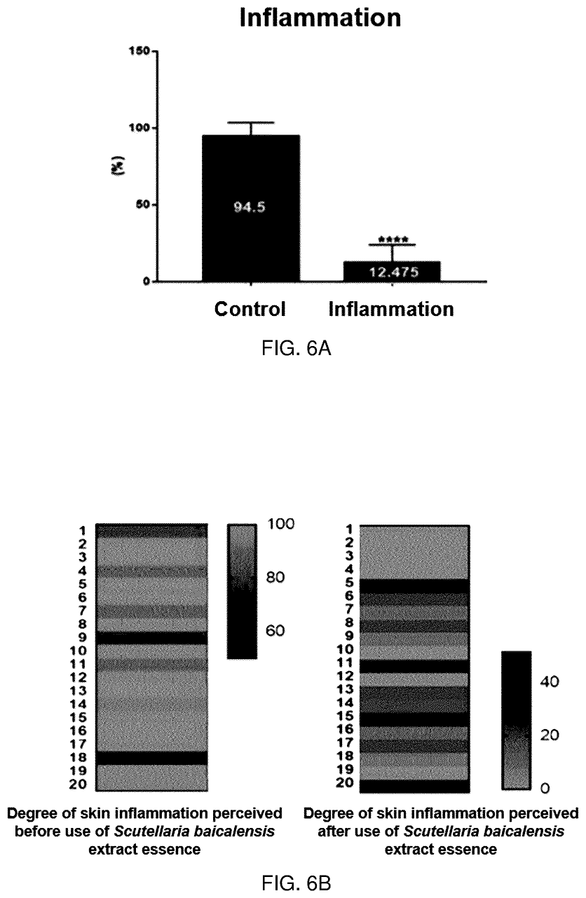

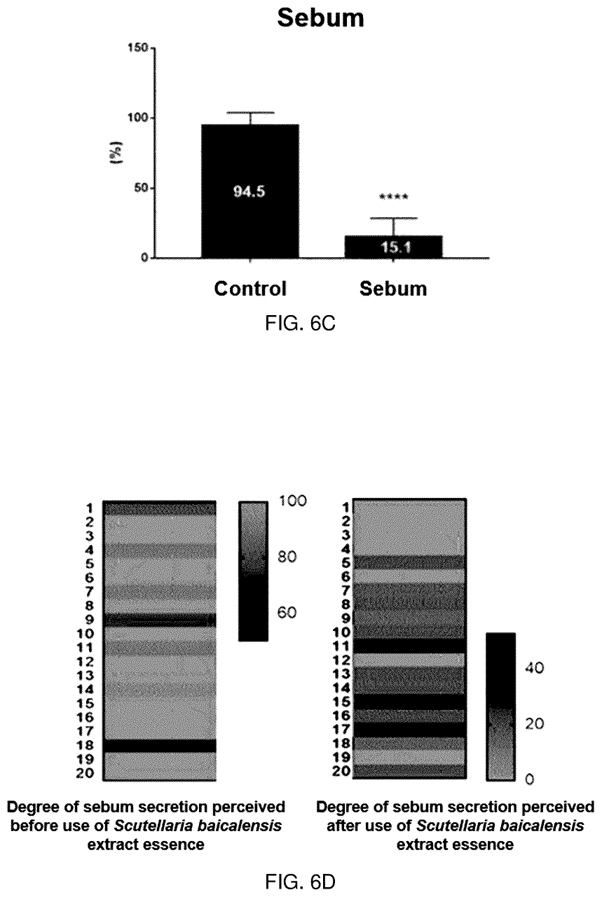

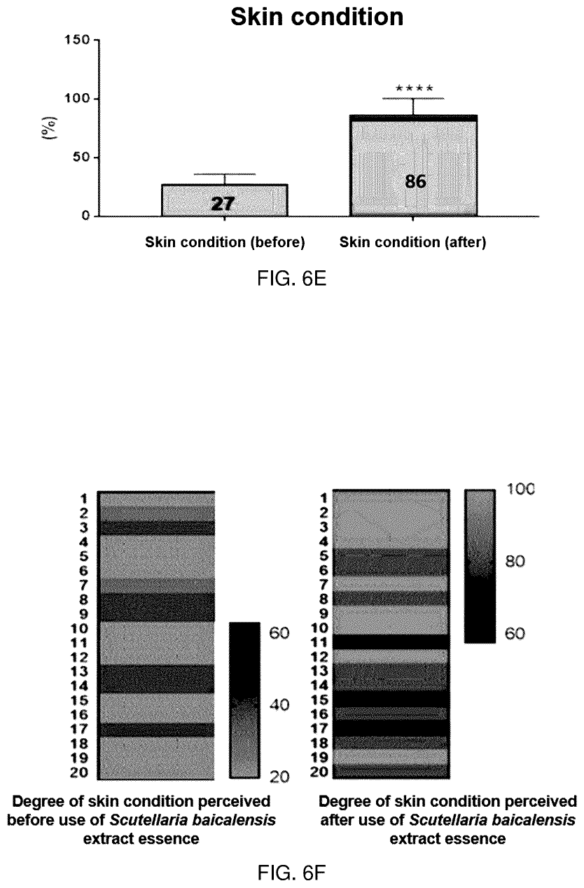

A sensory evaluation for investigating the skin inflammation alleviating effect, sebum secretion suppressing effect, and skin condition satisfaction increasing effect of the Scutellaria baicalensis extract essence was carried out on 20 subjects through a survey. Specifically, the degrees of skin inflammation, sebum secretion, and skin satisfaction before and 10 days after the use of the Scutellaria baicalensis extract essence were set as evaluation items. Each evaluation item was scored from 0 to 5 points, and the scores were converted based on 100% for evaluation. The results were analyzed using Prism 7 program (Graphpad, USA), with a paired T test (P<0.001), and shown in Table 3 and FIGS. 6A to 6F.

TABLE 3

| | Before application | After application | |
|---|---|---|---|
| | to skin | to skin | |
| Evaluation item | (mean ± s.d. (%)) | (mean ± s.d. (%)) | P-Value |
| Degree of skin | 94.5 ± 2.081 | 12.475 ± 2.597 | P < 0.001 |
| inflammation | | | |
| Degree of sebum | 94.5 ± 2.081 | 15.1 ± 3.01 | P < 0.001 |
| secretion | | | |
| Degree of skin | 27 ± 2.065 | 86 ± 3.277 | P < 0.001 |
| condition | | | |
| satisfaction | | | |

Example 6-1-1. Skin Inflammation Alleviating Effect of Scutellaria baicalensis Extract

As shown in Table 3 and FIGS. 6A and 6B, the subjects rated the degree of their skin inflammation as 94.5% before using the Scutellaria baicalensis extract essence, indicating that the subjects perceived their skin condition as having severe inflammation. However, after 10 days of using the Scutellaria baicalensis extract essence, the subjects rated the degree of skin inflammation as 12.5%, indicating that the degree of skin inflammation perceived by the subjects was reduced by about 7.6-fold. These results confirmed that the Scutellaria baicalensis extract essence significantly alleviated skin inflammation.

Example 6-1-2. Sebum Secretion Suppressing Effect of Scutellaria baicalensis Extract

As shown in Table 3 and FIGS. 6C and 6D, the subjects rated the degree of sebum secretion as 94.5% before using the Scutellaria baicalensis extract essence, indicating that the subjects perceived their sebum secretion condition as being severe. However, after 10 days of using the Scutellaria baicalensis extract essence, the subjects rated the degree of sebum secretion as 15.1%, indicating that the degree of sebum secretion perceived by the subjects was reduced by about 6.3-fold. These results confirmed that the Scutellaria baicalensis essence significantly suppressed sebum secretion.

Example 6-1-3. Skin Condition Satisfaction Increasing Effect of Scutellaria baicalensis Extract

As shown in Table 3 and FIGS. 6E and 6F, the subjects rated the degree of satisfaction on their skin condition as 27% before using the Scutellaria baicalensis extract essence, indicating a significant dissatisfaction on the skin conditions. However, after 10 days of using the Scutellaria baicalensis extract essence, the subjects rated the degree of satisfaction on their skin conditions as 86%, indicating that the degree of skin satisfaction was increased by about 3.1-fold.

### Example 6-2. Visual Evaluation

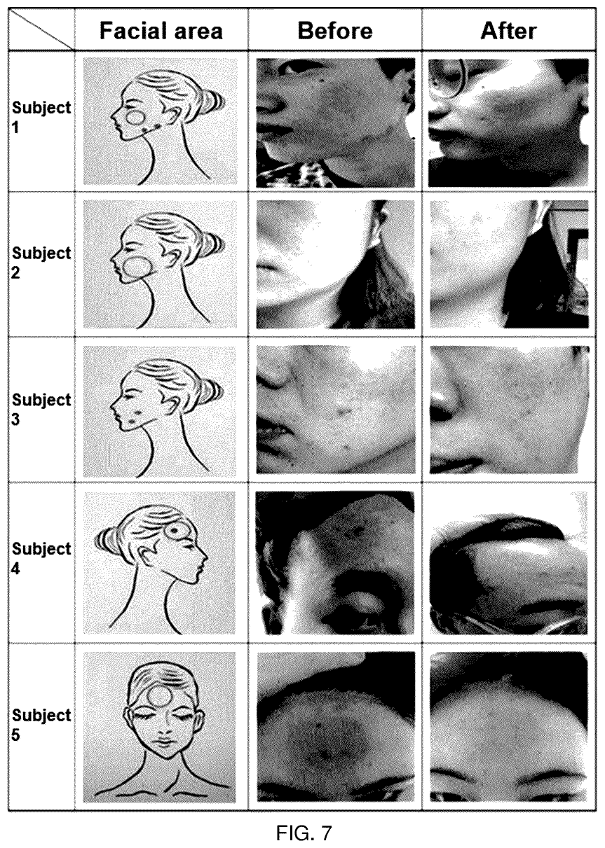

To observe the visual change before and after the application of the Scutellaria baicalensis extract, a visual evaluation was carried out through photography (FIG. 7). The Scutellaria baicalensis extract essence was applied twice a day, in the morning and evening, for 10 days, and the subjects took facial skin photographs using their own smartphones immediately after waking up in the morning.

Before using the Scutellaria baicalensis extract essence, subjects' facial areas with poor skin condition and many skin troubles were selected and individually marked on facial images. Specific inflamed areas were marked in dark red, and areas showing an overall fading of color or an improvement in skin condition were marked with a large circular shape.

As shown in FIG. 7, the comparison between images before and after using the Scutellaria baicalensis extract essence visually confirmed the mitigation of severe inflammations on the cheek, chin, or forehead, a clear skin tone, and an improvement in the entire skin condition.

### Example 6-3. Analysis by Magnifying Lens

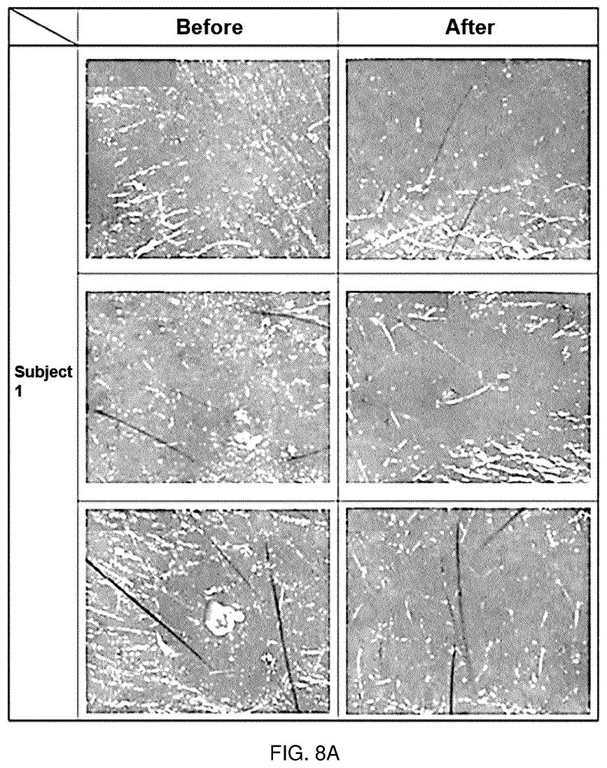

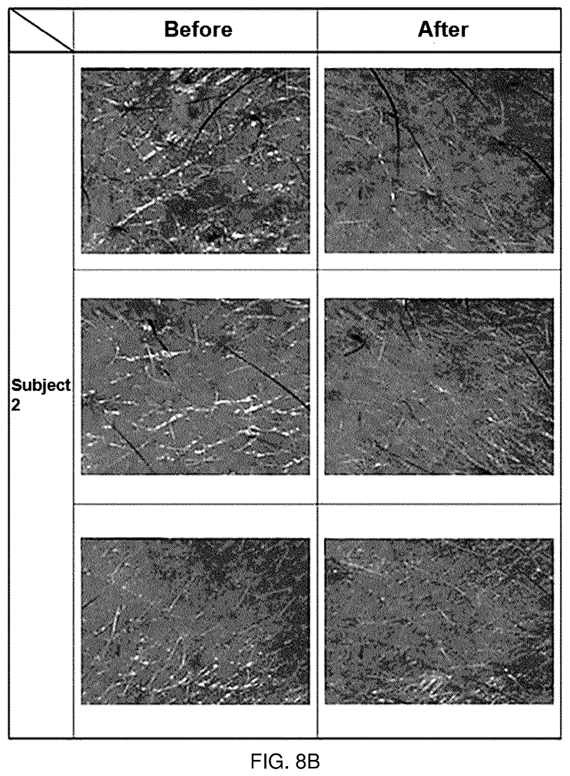

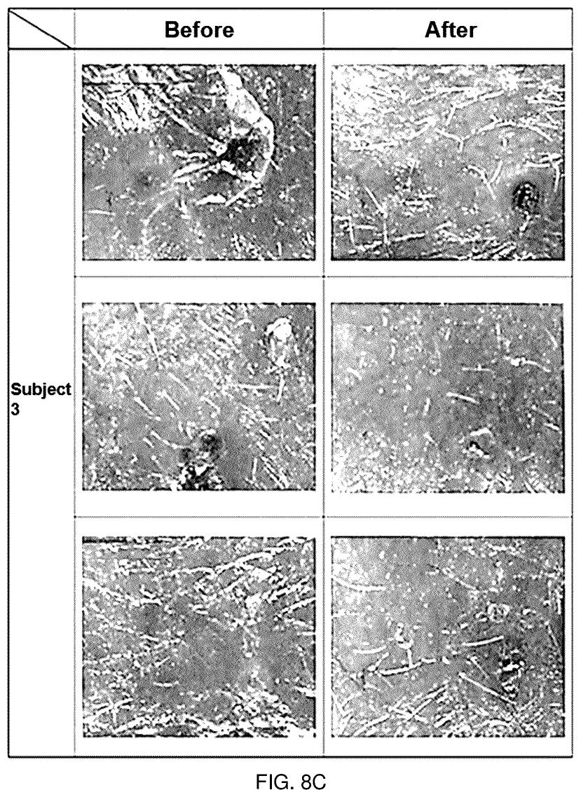

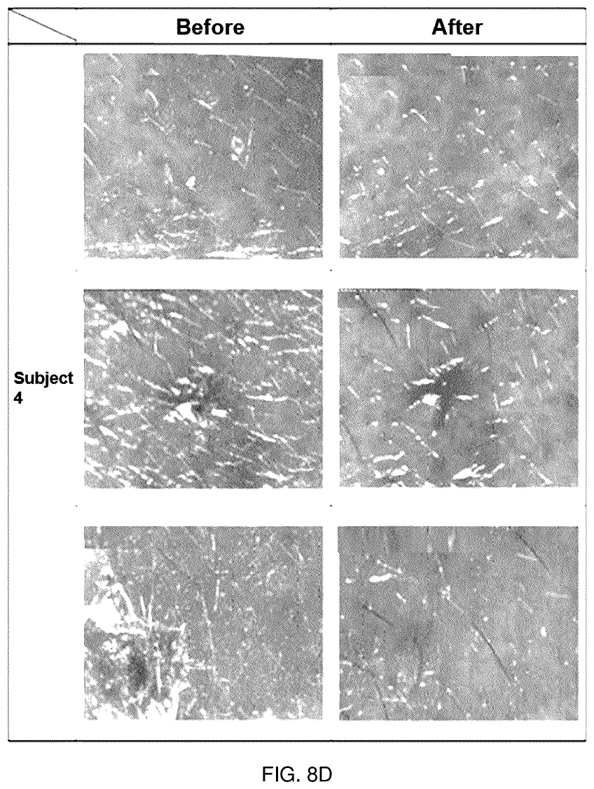

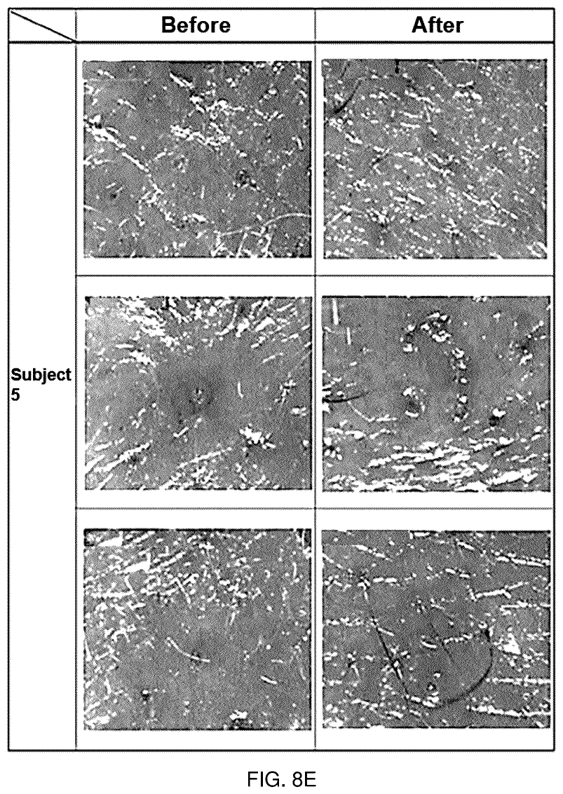

The skin change before and after the application of the Scutellaria baicalensis extract essence was analyzed using a magnifying lens. The Scutellaria baicalensis extract essence was applied twice a day, in the morning and evening, for 7 days, and the subjects took photographs at 200× magnification by using a magnifier for diagnosing the skin condition before and after the use (FIGS. 8A to 8E). The photographed area was 1 cm×1 cm.

As shown in FIGS. 8A to 8E, the comparison between images before and after using the Scutellaria baicalensis extract essence confirmed the improvements of inflammation, pus, and wounds, skin regeneration, improvement of flaky skin areas due to dryness, and brightening of the red skin tone.

### Example 6-4. Measurement and Analysis of Skin Sebum Contents

The skin sebum contents before and after the application of Scutellaria baicalensis extract essence were measured and analyzed.

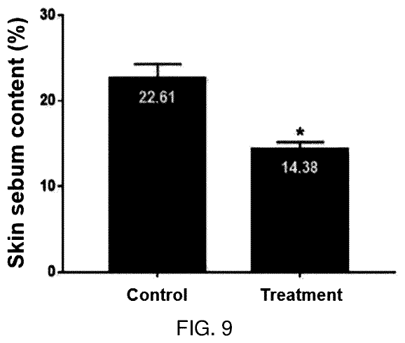

Before the application of the Scutellaria baicalensis extract essence, areas expected to have the most sebum, such as the forehead, nose bridge, and cheeks, were selected from the facial areas without makeup in 20 subjects, and the same areas, without the application of the Scutellaria baicalensis extract essence, were subjected to five or more times of measurements at time intervals by using the Skinalyzer Vcare Mode: SK-8 (China) sebum meter, and the measurement results were averaged. Thereafter, the Scutellaria baicalensis extract essence was applied twice daily, after facial washing in the morning and evening. On the 10th day, the sebum content was measured by the same method as before application, in order to obtain the results after the application (P<0.05). The results are shown in Table 4 and FIG. 9.

TABLE 4

| | Before application | After application | |
|---|---|---|---|
| | to skin | to skin | |
| Evaluation item | (mean ± s.d. (%)) | (mean ± s.d. (%)) | P-Value |
| Skin sebum content | 22.61 ± 1.67 | 14.38 ± 0.80 | 0.012 |

As shown in Table 4 and FIG. 9, the use of the Scutellaria baicalensis extract essence significantly reduced the sebum content, thereby alleviating skin oiliness.

Although the present disclosure has been described in detail with reference to the specific features, it will be apparent to those skilled in the art that this description is only for a preferred embodiment and does not limit the scope of the present disclosure.

1. A cosmetic composition for improvement of skin condition, comprising a Scutellaria baicalensis extract as an active ingredient.

2. The cosmetic composition of claim 1, wherein the Scutellaria baicalensis extract is obtained by extraction with any one selected from the group consisting of water, an organic solvent, and a mixture solvent thereof.

3. The cosmetic composition of claim 2, wherein the organic solvent is at least one selected from the group consisting of a C1 to C4 lower alcohol, petroleum ether, hexane, benzene, chloroform, methylene chloride, an ether, ethyl acetate, and acetone.

4. The cosmetic composition of claim 1, wherein the Scutellaria baicalensis extract is contained in a content of 0.01 wt % to 30 wt % relative to the total weight of the composition.

5. The cosmetic composition of claim 1, wherein the composition has an anti-pollution effect on harmful substances caused by air pollution.

6. The cosmetic composition of claim 1, wherein the composition has an anti-inflammatory effect.

7. The cosmetic composition of claim 1, wherein the composition has a sebum secretion suppressing effect.

8. The cosmetic composition of claim 7, wherein the composition alleviates a skin disease associated with excessive sebum secretion.

9. The cosmetic composition of claim 8, wherein the skin disease comprises acne or seborrheic dermatitis.

10. A method for improving skin condition, comprising:
applying an effective amount of a cosmetic composition comprising Scutellaria baicalensis extract as an active ingredient to a subject in need of skin condition improvement.

11. A method for alleviating a skin disease associated with excessive sebum secretion, comprising:
applying an effective amount of a cosmetic composition comprising Scutellaria baicalensis extract as an active ingredient to a subject in need of alleviation of skin disease.

12. The method of claim 11, wherein the skin disease comprises acne or seborrheic dermatitis.
